(19) United States
(12) Patent Application Publication
(10) Pub. No.: US 2024/0173090 A1
(43) Pub. Date: May 30, 2024

(54) SURGICAL SYSTEM AND SURGICAL SUPPORT METHOD

(71) Applicant: SONY GROUP CORPORATION, TOKYO (JP)

(72) Inventors: HIROYUKI SUZUKI, TOKYO (JP); ATSUSHI MIYAMOTO, TOKYO (JP); TOMOYUKI OOTSUKI, TOKYO (JP)

(21) Appl. No.: 18/551,753

(22) PCT Filed: Dec. 28, 2021
(86) PCT No.: PCT/JP2021/048962

§ 371 Date: Sep. 21, 2023

(30) Foreign Application Priority Data
Mar. 31, 2021 (JP) 2021-060413

(51) Int. Cl. A61B 34/37; A61B 34/20; A61B 90/14; A61B 90/20

(52) U.S. Cl. CPC A61B 34/37; A61B 34/20; A61B 90/14; A61B 90/20; A61B 2034/2068

(57) ABSTRACT

Provided is a surgical system that supports a surgical operation using an observation device and a surgical robot.

The surgical system includes: an observation device that observes an operative field; a surgical robot that supports a surgical tool; a fixing portion that fixes a relationship of relative position and posture between the observation device and the surgical robot; and a processing unit that performs processing for coordinate conversion between coordinates on a captured image of the observation device and coordinates of the surgical tool on the basis of the relationship of relative position and posture. The observation device includes a microscope and a pre-lens, and the fixing portion fixes a relationship of relative position and posture of the surgical robot with respect to the pre-lens.

### TECHNICAL FIELD

The technology disclosed in the present description (hereinafter, “the present disclosure”) relates to a surgical system and a surgical support method for supporting a surgical operation by applying a robotics technology.

### BACKGROUND ART

In general, surgical operation is a difficult task performed with sensory movement of an operator. In particular, in the case of surgery using a fine surgical tool in a small and fragile environment, such as ophthalmic surgery, it is necessary for the operator to perform a micron-order operation while suppressing tremor of the hands. Therefore, a surgical system that achieves restriction of tremor of the hands of an operator, absorption of the difference in skill between operators by operation support, and the like using a robotics technology is becoming widespread.

For example, a master slave system has been developed in which an operator operates a surgical tool while observing an operative field of a fundus portion through a microscope (see Non-Patent Document 1). According to this master slave system, the operator can perform the precise operation in the ophthalmic surgery by operating the slave robot supporting the surgical tool depending on an operation amount of the master robot held with the right hand (or the dominant hand).

Furthermore, there has been proposed a surgical system that controls scanning of a surgical laser beam on the basis of information of an optical coherence tomography (OCT) image to perform corneal incision, anterior capsule incision, and disruption of a crystalline lens in cataract surgery (see Patent Document 1).

In the surgical system as described above, an operator performs surgery while viewing an image of a target tissue captured by an observation device such as a microscope or an OCT. At that time, the operator operates the master robot (or the surgical tool) while imagining, in the brain, the spatial positional relationship between the captured image and the surgical tool. The operator needs to perform sufficient training to be proficient in hand-eye coordination between the surgical tool and the captured image.

### CITATION LIST

### Patent Document

Patent Document 1: Japanese Patent Application Laid-Open No. 2014-12201

### Non-Patent Document

Non-Patent Document 1: J. Wilson et al., “Intraocular robotic interventional surgical system (IRISS): Mechanical design, evaluation, and master-slave manipulation”, Int. J. Med. Robot. Comput. Assist. Surg., vol. 14, July 2017, doi:10.1002/rcs.1842.
Non-Patent Document 2: B. P. DeJong, J. E. Colgate, and M. A. Peshkin, “Improving teleoperation: reducing mental rotations and translations”, in IEEE International Conference on Robotics and Automation, 2004. Proceedings. ICRA'04.2004, 2004, vol. 4, pp. 3708-3714 Vol. 4.

### SUMMARY OF THE INVENTION

### Problems to be Solved by the Invention

An object of the present disclosure is to provide a surgical system and a surgical support method for supporting a surgical operation using an observation device such as a microscope or an OCT, and a surgical robot.

### Solutions to Problems

The present disclosure has been made in view of the above problems, and a first aspect thereof is a surgical system including:
an observation device that observes an operative field;
a surgical robot that supports a surgical tool;
a fixing portion that fixes a relationship of relative position and posture between the observation device and the surgical robot; and
a processing unit that performs processing for coordinate conversion between coordinates on a captured image of the observation device and coordinates of the surgical tool on the basis of the relationship of relative position and posture.

However, a “system” described here refers to a logical assembly of a plurality of apparatuses (or functional modules that implement specific functions), and it does not matter whether or not each of the apparatuses or functional modules is in a single housing.

For example, the observation device includes a microscope and a pre-lens, and the fixing portion is configured to fix a relationship of relative position and posture of the surgical robot with respect to the pre-lens.

Furthermore, for example, the fixing portion is configured to fix a relationship of relative position and posture of the surgical robot with respect to a retainer that has a marker and retains a state of the surgical site. The retainer is, for example, an eyelid speculum.

Furthermore, in a case where the surgical system includes a first surgical robot and a second surgical robot that support surgical tools, respectively, and the observation device includes a microscope and a pre-lens, the fixing portion may be configured to fix a relationship of relative position and posture of each of the first surgical robot and the second surgical robot with respect to the pre-lens.

Furthermore, a second aspect of the present disclosure is a surgical support method using a surgical system including an observation device that observes an operative field and a surgical robot that supports a surgical tool, a relationship of relative position and posture between the observation device and the surgical robot being fixed, the surgical support method including:
a processing step of performing processing for coordinate conversion between coordinates on a captured image of the observation device and coordinates of the surgical tool on the basis of the relationship of relative position and posture; and
a control step of controlling driving of the surgical robot on the basis of the coordinates of the surgical tool converted by the processing step.

### Effects of the Invention

According to the present disclosure, it is possible to provide a surgical system and a surgical support method for supporting execution of an accurate surgery by making an observation device and a surgical robot cooperate with each other.

Note that the effects described in the present description are merely examples, and the effects brought by the present disclosure are not limited thereto. Furthermore, the present disclosure may further provide additional effects in addition to the effects described above.

Still another object, feature, and advantage of the present disclosure will become clear by further detailed description with reference to an embodiment as described later and the attached drawings.

### BRIEF DESCRIPTION OF DRAWINGS

FIG. 1 is a diagram illustrating an example of a functional configuration of a surgical system 100 of a master-slave system.

FIG. 2 is a diagram illustrating a general layout (surface of an eyeball) of fundus surgery.

FIG. 3 is a diagram illustrating a general layout (cross section of an eyeball) of fundus surgery.

FIG. 4 is a diagram illustrating a layout of fundus surgery using a surgical robot.

FIG. 5 is a diagram illustrating an arrangement example (first embodiment) of an observation device 500 and a surgical robot 510.

FIG. 6 is a diagram illustrating an arrangement example (second embodiment) of an observation device 600 and a surgical robot 610.

FIG. 7 is an arrow view illustrating an arrangement example (second embodiment) of the observation device 600 and the surgical robot 610.

FIG. 8 is a diagram illustrating an arrangement example (third embodiment) of an observation device 800, a first surgical robot 810, and a second surgical robot 820.

### MODE FOR CARRYING OUT THE INVENTION

Hereinafter, the present disclosure will be described in the following order with reference to the drawings.
A. Surgical system
B. Problems of surgical operation using surgical robot and outline of present disclosure
C. First example
D. Second example
E. Third example
F. Effects

### A. SURGICAL SYSTEM

In the present description, an embodiment in which the present disclosure is mainly applied to a surgical system of a master-slave system will be primarily described. In such a surgical system, a user such as an operator performs an operation on the master side, and performs surgery on the slave side by controlling driving of a robot according to the user's operation. Examples of the purpose of incorporating the robotics technology into the surgical system include restriction of tremor of the hands of an operator, operation support, absorption of a difference in skill between operators, and execution of surgery from a remote site.

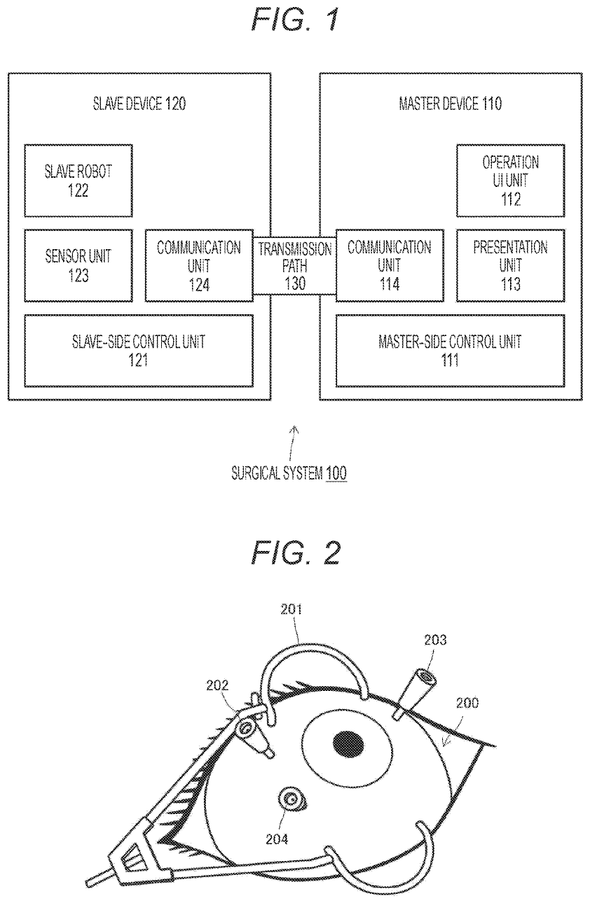

FIG. 1 illustrates an example of a functional configuration of a surgical system 100 of a master-slave system. The surgical system 100 illustrated includes a master device 110 in which a user (operator) instructs work such as surgery and a slave device 120 that performs surgery according to an instruction from the master device 110. As the surgery here, retinal surgery is mainly assumed. The master device 110 and the slave device 120 are interconnected via a transmission path 130. The transmission path 130 is preferably capable of performing signal transmission with low latency using a medium such as an optical fiber.

The master device 110 includes a master-side control unit 111, an operation user interface (UI) unit 112, a presentation unit 113, and a master-side communication unit 114. The master device 110 operates under the overall control of the master-side control unit 111.

The operation UI unit 112 includes a device to which a user (operator or the like) inputs an instruction for a slave robot 112 (described later) that operates a surgical tool such as forceps in the slave device 120. The operation UI unit 112 includes, for example, a dedicated input device such as a controller or a joystick, and a general-purpose input device such as a GUI screen to which a mouse operation or a touch operation with a fingertip is input. Furthermore, a “medical device” configured by supporting a gripping interface by a parallel link as disclosed in Patent Document 2 can be used as the operation UI unit 112.

The presentation unit 113 presents information regarding the surgery performed on the slave device 120, to a user (operator) operating the operation UI unit 112, on the basis of sensor information mainly acquired by a sensor unit 123 (described later) on the slave device 120 side.

For example, in a case where the sensor unit 123 is equipped with an observation device such as an RGB camera or an OCT for capturing a microscopic image for observing the surface of an affected site, or is equipped with an interface for capturing an image captured by these observation devices, and the image data is transferred to the master device 110 with low latency via the transmission path 130, the presentation unit 113 displays on the screen a real-time microscopic image or OCT image of the affected site using a monitor display or the like.

Furthermore, in a case where the sensor unit 123 is equipped with a function of measuring an external force and a moment acting on the surgical tool operated by the slave robot 112, and such force sense information is transferred to the master device 110 with low latency via the transmission path 130, the presentation unit 113 presents the force sense to the user (operator). For example, the presentation unit 113 may present the force sense to the user (operator) using the operation UI unit 112.

The master-side communication unit 114 performs a signal transmission/reception process with the slave device 120 via the transmission path 130 under the control of the master-side control unit 111. For example, in a case where the transmission path 130 includes an optical fiber, the master-side communication unit 114 includes an electro-optical conversion unit that converts an electric signal transmitted from the master device 110 into an optical signal, and a photoelectric conversion unit that converts an optical signal received from the transmission path 130 into an electric signal.

The master-side communication unit 114 transfers an operation command for the slave robot 122 input by the user (operator) via the operation UI unit 112 to the slave device 120 via the transmission path 130. Furthermore, the master-side communication unit 114 receives the sensor information transmitted from the slave device 120 via the transmission path 130.

On the other hand, the slave device 120 includes a slave-side control unit 121, a slave robot 122, a sensor unit 123, and a slave-side communication unit 124. The slave device 120 performs an operation depending on an instruction from the master device 110 under the overall control of the slave-side control unit 121.

The slave robot 122 is, for example, an arm type robot having a multi-link structure, and a surgical tool such as forceps is mounted as an end effector on a tip end (or the distal end). The slave-side control unit 121 interprets an operation command transmitted from the master device 110 via the transmission path 130, converts the operation command into a drive signal of an actuator that drives the slave robot 122, and outputs the drive signal. Then, the slave robot 122 operates on the basis of the drive signal from the slave-side control unit 121.

The sensor unit 123 includes a plurality of sensors for detecting a situation in an affected site of the surgery performed by the slave robot 122 or the slave robot 122, and further includes an interface for acquiring sensor information from various sensor devices installed in an operating room.

For example, the sensor unit 123 includes a force torque sensor (FTS) for measuring an external force and a moment applied during surgery on a surgical tool mounted on the tip end (distal end) of the slave robot 122.

Furthermore, the sensor unit 123 is provided with an interface through which, during the surgery, the slave robot 122 captures a microscopic image of the surface of an affected site or an OCT image obtained by scanning a cross section of the affected site (eyeball).

The slave-side communication unit 124 performs a signal transmission/reception process with the master device 110 via the transmission path 130 under the control of the slave-side control unit 121. For example, in a case where the transmission path 130 includes an optical fiber, the slave-side communication unit 124 includes an electro-optical conversion unit that converts an electric signal transmitted from the slave device 120 into an optical signal, and a photoelectric conversion unit that converts an optical signal received from the transmission path 130 into an electric signal.

The slave-side communication unit 124 transfers the force sense data of a surgical tool acquired by the sensor unit 123, a microscopic image of the affected site, the OCT image obtained by scanning the cross section of an affected site, and the like to the master device 110 via the transmission path 130. Furthermore, the slave-side communication unit 124 receives an operation command for the slave robot 122 transmitted from the master device 110 via the transmission path 130.

### B. PROBLEMS OF SURGICAL OPERATION USING SURGICAL ROBOT AND OUTLINE OF PRESENT DISCLOSURE

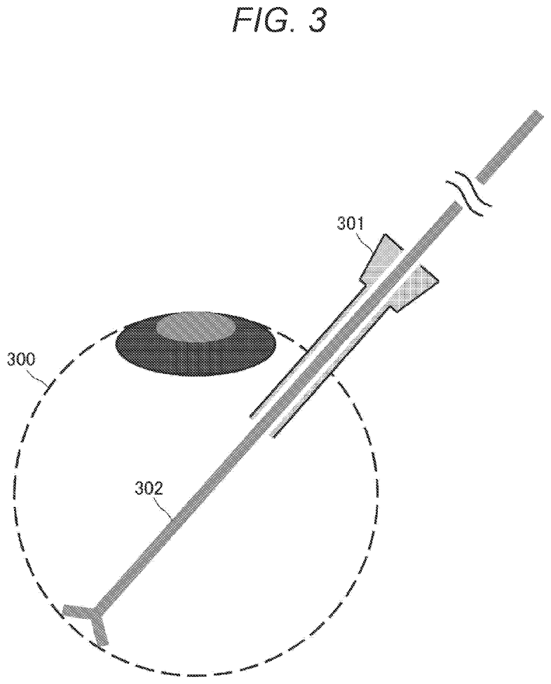

FIGS. 2 and 3 illustrate a general layout of fundus surgery (retinal surgery or the like). However, FIG. 2 illustrates a surface of an eyeball, and FIG. 3 illustrates a cross section of an eyeball cut along a plane on which the trocar and the surgical tool (forceps) pass.

As illustrated in FIG. 2, an eyelid speculum 201 is attached to an eyeball 200 which is a subject eye, and fixed so as to prevent the eyelid from closing. Then, trocars 202 to 204 is inserted into a plurality of places (3 places in an example illustrated in FIG. 2) on the surface of the eyeball 200. The trocars 202 to 204 have a tube with a small diameter into which a surgical tool such as forceps is inserted.

As illustrated in FIG. 3, a trocar 301 having a tube with a small diameter is stuck on the surface of an eyeball 300, and forceps 302 are inserted into the eyeball 300 via the trocar 301 and further reach the eye fundus to perform retinal surgery. Note that the operator (alternatively, the slave robot 122 remotely controlled by the operator via the master device 110) always considers that surgery is performed with as small a load as possible with respect to the vicinity of an intersection (also referred to as “insertion point”) between the trocar 301 and the surface of the eyeball 300 for the convenience of minimally invasive surgery. Therefore, it is ideal to perform an operation of making an impulse generated at the insertion point zero by pivotally operating the forceps 302 with the insertion point as a fulcrum by a remote center of motion (RCM) mechanism of the slave robot 122.

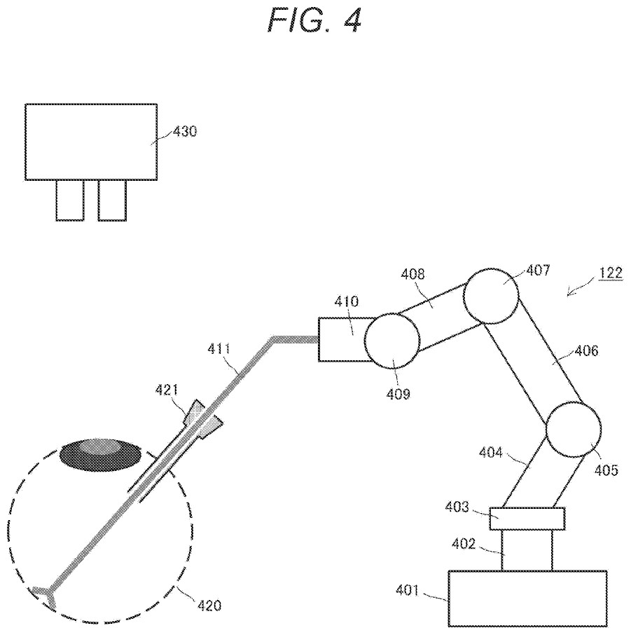

FIG. 4 illustrates a layout of fundus surgery using a surgical robot. The surgical robot corresponds to the slave robot 122 in the surgical system 100 illustrated in FIG. 1. In an example illustrated in FIG. 4, the surgical robot includes a base portion 401 rigidly fixed to a mechanical ground (M-GND), a link 402 attached perpendicularly to the base portion 401, and a robot arm attached to an upper end of the link 402 via a joint 403. It is assumed that the joint 403 has a rotational degree of freedom about a yaw axis.

In the example illustrated in FIG. 4, the robot arm has a serial link structure, and includes links 404, 406, 408, and 410, a joint 405 that hingedly couples the link 404 and the link 406, a joint 407 that hingedly couples the link 406 and the link 408, and a joint 409 that hingedly couples the link 408 and the link 410. Each of the joints 405, 407, and 409 has a rotational degree of freedom about a roll axis (alternatively, about an axis orthogonal to the yaw axis). Then, a surgical tool 411 such as forceps is attached to the link 410 at the distal end.

An eyelid speculum (not illustrated in FIG. 4) is attached to the eyeball 420 which is a subject eye so as to prevent the eyelid from closing. Then, a trocar 421 is inserted into the surface of the eyeball 420. FIG. 4 illustrates a cross section of eyeball 420 cut along a plane on which the trocar 421 passes. The surgical tool 411 mounted on the distal end of the robot arm is inserted into the eyeball 420 via one trocar 421.

Note that, it is sufficient that a movable range of the surgical tool 411 required for the fundus operation be small, so that it is assumed that the robot arm is a microrobot having a total length or a total height of about several centimeters and a mass of about several grams to several tens grams.

Furthermore, for the information of the eyeball 420, an observation device (a stereo video microscope in the example illustrated in FIG. 4) 430 such as a microscope or an OCT is installed. The observation device 430 corresponds to the sensor unit 123 in the surgical system 100 illustrated in FIG. 1.

The operator operates the surgical tool 411 while observing an operative field such as the surface and the fundus of the eyeball via the captured image of the observation device 430. In a case where the surgical system 100 is used, the slave robot 122 supporting the surgical tool 411 operates depending on an operation amount of the operation UI unit 112 operated by the operator with the right hand (or the dominant hand), to perform the fundus surgery.

At that time, the operator operates the operation UI unit 112 while imagining, in the brain, the spatial positional relationship between the captured image of the observation device 430 and the surgical tool 411. The operator needs to perform sufficient training to be proficient in hand-eye coordination between the surgical tool 411 and the captured image. In general, the relationship of relative position and posture between the captured image of the observation device 430 and the robot arm is unknown. In such a case, even if the operator tries to instruct the slave robot 122 to operate the surgical tool 411 through an operation of the operation UI unit 112 on the basis of the captured image of the observation device 430, an operation amount of the operation UI unit 112 based on the captured image and the motion of the surgical tool 411 intended by the operator deviate from each other, making it difficult to perform a precise surgery. Furthermore, in a case where many components are mechanically connected between the slave robot 122 and the observation device 430, the accuracy of the position and posture of the surgical tool 411 is also affected by the deflection of the robot arm and the processing accuracy of the components.

Therefore, in the present disclosure, the relationship of relative position and posture between the observation device that observes an operative field and the robot arm that supports a surgical tool is fixed. As a result, for example, an operation amount of the operation UI unit 112 on a captured image of the observation device on the master side can be coordinate-transformed into a movement of the distal end (alternatively, the surgical tool 411 mounted on the distal end) of the robot arm. Therefore, according to the present disclosure, even if the operator is not proficient in hand-eye coordination, the operator can perform accurate surgery by making the observation device and the surgical system of the master-slave system cooperate with each other.

### C. FIRST EXAMPLE

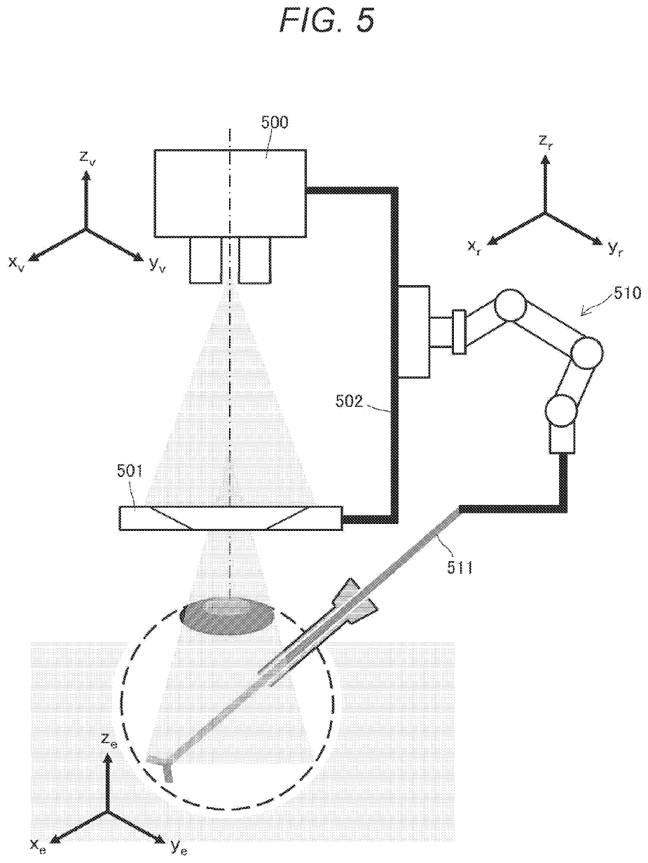

FIG. 5 illustrates an arrangement example of an observation device 500 and a surgical robot 510 according to the first example of the present disclosure. Specifically, an arrangement example of the observation device 500 and the surgical robot 510 in a case of being applied to fundus surgery is illustrated.

The observation device 500 is, for example, a stereo video microscope equipped with an OCT, and corresponds to the sensor unit 123 in the surgical system 100 illustrated in FIG. 1. The observation device 500 is disposed at a position where the subject eye is observed from above. There are two types of microscopes, one of which is a type with a pre-lens disposed between the subject eye and a focal position of an objective lens, and the other is a type without a pre-lens. The pre-lens has a purpose of, for example, focusing illumination light to illuminate the inside of the eye. A wide-angle observation lens is widely used as the pre-lens in retinal vitreous surgery, and a gonioscope is widely used as the pre-lens in minimally invasive glaucoma surgery (MIGS) for treating a corner angle. In an example illustrated in FIG. 5, the observation device 500 is a stereo video microscope having a pre-lens 501.

Furthermore, the surgical robot 510 corresponds to the slave robot 122 in the surgical system 100 illustrated in FIG. 1. In the example illustrated in FIG. 5, the surgical robot 510 includes a robot arm having a serial link structure (see, for example, FIG. 4), and is equipped with a surgical tool 511 on the distal end. It is sufficient that a movable range of the surgical tool 511 required for the fundus operation be small, so that it is assumed that the surgical robot 510 is a microrobot having a total length or a total height of about several centimeters and a mass of about several grams to several tens grams.

Then, in the example illustrated in FIG. 5, the surgical robot 510 is attached onto the pre-lens 501. Assuming that the surgical robot 510 is a microrobot, it is sufficiently possible to install the surgical robot 510 on the pre-lens 501. In FIG. 5, it is assumed that the surgical robot 510 is fixed to the pre-lens 501 by a fixing portion 502. However, a method for fixing the surgical robot 510 to the pre-lens 501 is not particularly limited.

In the first place, the positional relationship between the observation device 500 and the pre-lens 501 is known. Then, since the surgical robot 510 is attached onto the pre-lens 501, the positional relationship between the captured image of the observation device 500 and the surgical robot 510 is known. Assuming that the coordinate system of the captured image of the observation device 500 is (xᵥ, yᵥ, zᵥ) and the coordinate system of the surgical robot 510 is (xᵣ, yᵣ, zᵣ), the coordinate system (xᵥ, yᵥ, zᵥ) of the captured image of the observation device 500 can be converted into the coordinate system (xᵣ, yᵣ, zᵣ) of the surgical robot 510 using a conversion matrix A₁ as illustrated in the following equation (1). Since the positional relationship between the observation device 500 and the surgical robot 510 is known, the conversion matrix A₁ can be obtained.

[Equation 1]

(xᵣ,yᵣ,zᵣ,1)=(xᵥ,yᵥ,zᵥ,1)A₁ (1)

On the other hand, since the configuration information of the surgical robot 510 (the configuration information of each link and joint of the robot arm) and the configuration information of the surgical tool 511 attached to the distal end of the surgical robot 510 are known, the positional relationship between the surgical robot 510 and the surgical tool 511 is known. Assuming that the coordinate system of the tip end of the surgical tool 511 is (xₑ, yₑ, zₑ), the coordinate system (xᵣ, yᵣ, zᵣ) of the surgical robot 510 can be converted into the coordinate system (xₑ, yₑ, zₑ) of the tip end of the surgical tool 511 using a conversion matrix A₂ as illustrated in the following equation (2). The conversion matrix A₂ can be obtained on the basis of the configuration information of the surgical robot 510 and the configuration information of the surgical tool 511.

[Equation 2]

(xₑ,yₑ,zₑ,1)=(xᵣ,yᵣ,zᵣ,1)A₂ (2)

Therefore, the coordinate relationship between the captured image of the observation device 500 and the tip end of the surgical tool 511 is determined as illustrated in the following equation (3).

[Equation 3]

(xₑ,yₑ,zₑ,1)=(xᵥ,yᵥ,zᵥ,1)A₁A₂ (3)

A case where the arrangement of the observation device 500 and the surgical robot 510 as illustrated in FIG. 5 is applied to the surgical system 100 will be described. The captured image of the observation device 500 is displayed on a screen of the monitor display included in the presentation unit 113. The operator gives instruction on an operation amount of the surgical tool 511 on the captured image displayed on the monitor screen using the operation UI unit 112. The master-side control unit 111 transfers information on the amount of operation (xᵥ, yᵥ, zᵥ) expressed in the coordinate system of the captured image of the observation device 500 to the slave device 120 via the transmission path 130. Then, on the side of the slave device 120, in a case where the operation amount (xᵥ, yᵥ, zᵥ) is converted into the coordinate system (xₑ, yₑ, zₑ) of the tip end of the surgical tool 511 on the basis of the above equation (3), the operation amount is only required to be further converted into a command value of the surgical robot 510 (joint angle of each joint of the robot arm) for achieving the movement of the tip end of the surgical tool 511 corresponding to the operation amount of the operation UI unit 112 by inverse kinematics operation, to control the driving of the surgical robot 510.

The hand-eye coordination in the surgical system 100 in this case is that the operator visually views the captured image of the observation device 500, accurately grasps the position information of the tip end of the surgical tool 511 with respect to the captured image, predicts a trajectory of the tip end of the surgical tool 511, and performs operation using the operation UI unit 112. As described above, according to the first example of the present disclosure, the operator views the captured image of the observation device 500 and performs the input to the operation UI unit 112, and the operation of the surgical tool 511 by the surgical robot 510 can be performed smoothly. That is, according to the first example of the present disclosure, even if the operator is not fully trained and proficient in hand-eye coordination, the operator can perform accurate surgery by making the observation device and the surgical system of the master-slave system cooperate with each other.

A condition under which the optimum hand-eye coordination is established can be defined as in the following equation (4) (see Non-Patent Document 2). From the above equation (3), it can be seen that, according to the first example of the present disclosure, the conditional equation (4) is satisfied.

[Equation 4]

^{C}_{S}R=^{S}_{M}R (4)
^{C}_{S}R: Posture of tip end of surgical tool viewed from observation device (Roll, Pitch, Yaw)
^{D}_{M}R: Posture of operation UI unit viewed from monitor screen (Roll, Pitch, Yaw)

### D. SECOND EXAMPLE

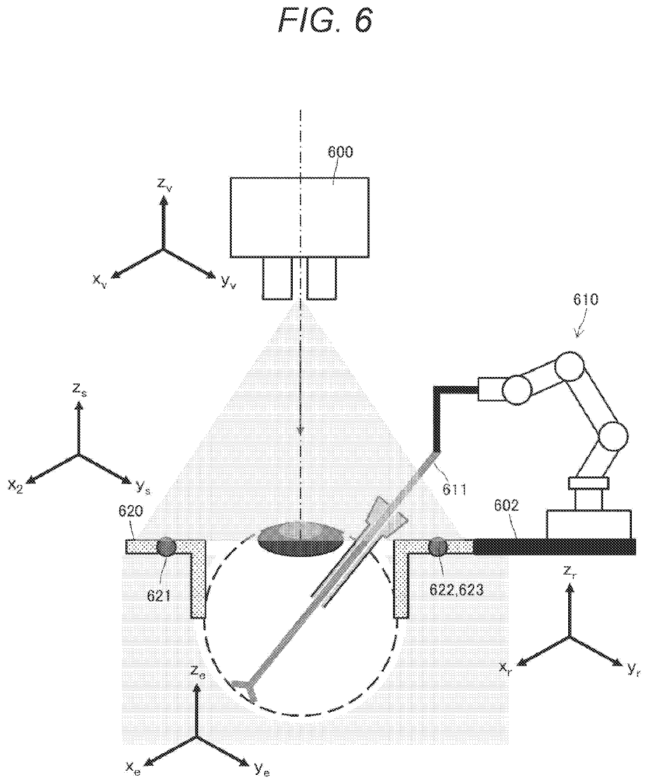

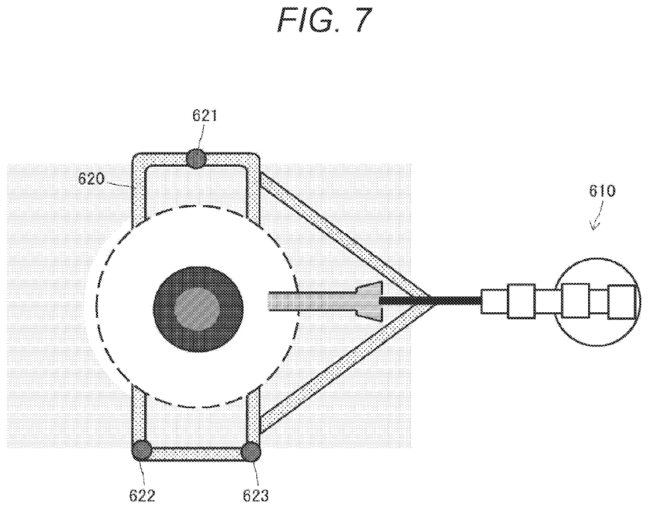

FIG. 6 illustrates an arrangement example of an observation device 600 and a surgical robot 610 according to the second example of the present disclosure. Specifically, an arrangement example of the observation device 600 and the surgical robot 610 in a case of being applied to fundus surgery is illustrated. Furthermore, for reference, FIG. 7 illustrates an arrow view of an arrangement of the observation device 600 and the surgical robot 610 as viewed from above.

The observation device 600 is, for example, a stereo video microscope equipped with an OCT, and corresponds to the sensor unit 123 in the surgical system 100 illustrated in FIG. 1. The observation device 600 is disposed at a position where the subject eye is observed from above. Furthermore, an eyelid speculum 620 is attached to the subject eye, and is fixed so as to prevent the eyelid from closing. The eyelid speculum 620 has visual markers 621, 622, and 623 at three locations. Then, a positional relationship (size and shape of a triangle formed by the markers 621, 622, and 623) among the markers 621, 622, and 623 is known in the surgical system 100. Therefore, the observation device 600 simultaneously images the operative field and the markers 621, 622, and 623 attached to the eyelid speculum 620, and thus the relationship of relative position and posture between the observation device 600 and the eyelid speculum 620 can be calculated on the basis of the positional relationship among the markers 621, 622, and 623 on the captured image.

Furthermore, the surgical robot 610 corresponds to the slave robot 122 in the surgical system 100 illustrated in FIG. 1. In the example illustrated in FIGS. 6 and 7, the surgical robot 610 includes a robot arm having a serial link structure (see, for example, FIG. 4), and is equipped with a surgical tool 611 on the distal end. It is sufficient that a movable range of the surgical tool 611 required for the fundus operation be small, so that it is assumed that the surgical robot 610 is a microrobot having a total length or a total height of about several centimeters and a mass of about several grams to several tens grams.

Then, in the example illustrated in FIGS. 6 and 7, the surgical robot 610 is attached onto the eyelid speculum 620. Assuming that the surgical robot 610 is a microrobot, it is sufficiently possible to install the surgical robot 610 on the eyelid speculum 620. In FIGS. 6 and 7, it is assumed that the surgical robot 610 is fixed to the eyelid speculum 620 by a fixing portion 602. However, a method for fixing the surgical robot 610 to the eyelid speculum 620 is not particularly limited.

Assuming that the coordinate system of the captured image of the observation device 600 is (xᵥ, yᵥ, zᵥ) and the coordinate system of the eyelid speculum 620 is (xₛ, yₛ, zₛ), the coordinate system (xᵥ, yᵥ, zᵥ) of the captured image of the observation device 500 can be converted into the coordinate system (xₛ, yₛ, zₛ) of the eyelid speculum 620 using a conversion matrix B₁ as illustrated in the following equation (5). As described above, the observation device 600 simultaneously images the operative field and the markers 621, 622, and 623 attached to the eyelid speculum 620, and thus the conversion matrix B₁ can be obtained on the basis of the relationship of relative position and posture between the observation device 600 and the eyelid speculum 620 calculated on the basis of the positional relationship among the markers 621, 622, and 623 on the captured image.

[Equation 5]

(xₛ,yₛ,zₛ,1)=(xᵥ,yᵥ,zᵥ,1)B₁ (5)

Then, since the surgical robot 610 is attached onto the eyelid speculum 620, the positional relationship between the eyelid speculum 620 and the surgical robot 610 is known. Assuming that the coordinate system of the surgical robot 610 is (xᵣ, yᵣ, zᵣ), the coordinate system (xᵥ, yᵥ, zᵥ) of the captured image of the eyelid speculum 620 can be converted into the coordinate system (xᵣ, yᵣ, zᵣ) of the surgical robot 610 using a conversion matrix B₂ as illustrated in the following equation (6). Since the positional relationship between the eyelid speculum 620 and the surgical robot 610 is known, the conversion matrix B₂ can be obtained.

[Equation 6]

(xᵣ,yᵣ,zᵣ,1)=(xₛ,yₛ,zₛ,1)B₂ (6)

Furthermore, since the configuration information of the surgical robot 610 (the configuration information of each link and joint of the robot arm) and the configuration information of the surgical tool 611 attached to the distal end of the surgical robot 610 are known, the positional relationship between the surgical robot 610 and the surgical tool 611 is known. Assuming that the coordinate system of the tip end of the surgical tool 611 is (xₑ, yₑ, zₑ), the coordinate system (xᵣ, yᵣ, zᵣ) of the surgical robot 610 can be converted into the coordinate system (xₑ, yₑ, zₑ) of the tip end of the surgical tool 611 using a conversion matrix B₃ as illustrated in the following equation (7). The conversion matrix B₃ can be obtained on the basis of the configuration information of the surgical robot 510 and the configuration information of the surgical tool 511.

[Equation 7]

(xₑ,yₑ,zₑ,1)=(xᵣ,yᵣ,zᵣ,1)B₃ (7)

Therefore, the coordinate relationship between the captured image of the observation device 600 and the tip end of the surgical tool 611 is determined as illustrated in the following equation (8).

[Equation 8]

(xₑ,yₑ,zₑ,1)=(xᵥ,yᵥ,zᵥ,1)B₁B₂B₃ (8)

A case where the arrangement of the observation device 600 and the surgical robot 610 as illustrated in FIG. 6 is applied to the surgical system 100 will be described. The captured image of the observation device 600 is displayed on the screen of the monitor display included in the presentation unit 113. The operator gives instruction on an operation amount of the surgical tool 511 on the captured image displayed on the monitor screen using the operation UI unit 112. The master-side control unit 111 transfers information on the amount of operation (xᵥ, yᵥ, zᵥ) expressed in the coordinate system of the captured image of the observation device 600 to the slave device 120 via the transmission path 130. Then, on the side of the slave device 120, in a case where the operation amount (xᵥ, yᵥ, zᵥ) is converted into the coordinate system (xₑ, yₑ, zₑ) of the tip end of the surgical tool 611 on the basis of the above equation (8), the operation amount is only required to be further converted into a command value of the surgical robot 610 (joint angle of each joint of the robot arm) for achieving the movement of the tip end of the surgical tool 611 corresponding to the operation amount of the operation UI unit 112 by inverse kinematics operation, to control the driving of the surgical robot 610.

As described above, also in the second example of the present disclosure, the operator views the captured image of the observation device 600 and performs the input to the operation UI unit 112, and the operation of the surgical tool 611 by the surgical robot 610 can be performed smoothly.

From the above equation (8), it can be seen that, according to the second example of the present disclosure, the conditional equation (4) described above in which the optimum hand-eye coordination is established is satisfied. Therefore, also in the second example of the present disclosure, even if the operator is not fully trained and proficient in hand-eye coordination, the operator can perform accurate surgery by making the observation device and the surgical system of the master-slave system cooperate with each other.

### E. THIRD EXAMPLE

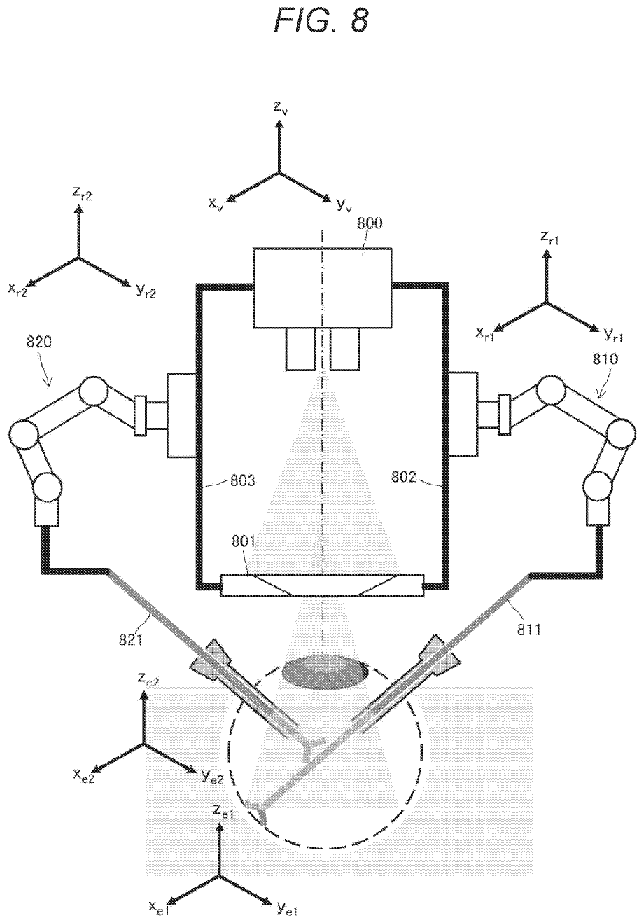

FIG. 8 illustrates an arrangement example of an observation device 800 and two surgical robots 810 and 820 according to the third example of the present disclosure. Specifically, an arrangement example of the observation device 800, a first surgical robot 810, and a second surgical robot 820 in a case of being applied to fundus surgery is illustrated.

The observation device 800 is, for example, a stereo video microscope equipped with an OCT, and corresponds to the sensor unit 123 in the surgical system 100 illustrated in FIG. 1. The observation device 800 is disposed at a position where the subject eye is observed from above. There are two types of microscopes: a type in which a pre-lens is disposed between the subject eye and a focal position of an objective lens, and a type in which no pre-lens is disposed (described above), and the observation device 800 is a stereo-video microscope having a pre-lens 801.

The first surgical robot 810 and the second surgical robot 820 correspond to the slave robot 122 in the surgical system 100 illustrated in FIG. 1. In the example illustrated in FIG. 8, both the first surgical robot 810 and the second surgical robot 820 include a robot arm having a serial link structure (see, for example, FIG. 4). However, the first surgical robot 810 and the second surgical robot 820 do not need to have the same configuration. The first surgical robot 810 is equipped with a surgical tool 811 on the distal end, and the second surgical robot 820 is equipped with a surgical tool 812 on the distal end. It is sufficient that movable ranges of the surgical tools 811 and 812 required for the fundus operation be small, so that it is assumed that both the first surgical robot 810 and the second surgical robot 820 are microrobots having a total length or a total height of about several centimeters and a mass of about several grams to several tens grams.

Then, in the example illustrated in FIG. 8, both the first surgical robot 810 and the second surgical robot 820 are attached onto the pre-lens 801. Assuming that the first surgical robot 810 and the second surgical robot are microrobots, it is sufficiently possible to install the first surgical robot 810 and the second surgical robot 820 on the pre-lens 801. In FIG. 8, it is assumed that the first surgical robot 810 and the second surgical robot 820 are fixed to the pre-lens 801 by fixing portions 802 and 803, respectively. However, a method for fixing the first surgical robot 810 and the second surgical robot 820 to the pre-lens 801 is not particularly limited.

In the first place, the positional relationship between the observation device 800 and the pre-lens 801 is known. Then, since the first surgical robot 810 and the second surgical robot 820 are attached onto the pre-lens 801, the positional relationship among the captured image of the observation device 800, the first surgical robot 810, and the second surgical robot 820 is known. Assuming that the coordinate system of the captured image of the observation device 800 is (xᵥ, yᵥ, zᵥ) and the coordinate system of the first surgical robot 80 is (xᵣ₁, yᵣ₁, zᵣ₁), the coordinate system (xᵥ, yᵥ, zᵥ) of the captured image of the observation device 800 can be converted into the coordinate system (xᵣ, yᵣ, zᵣ) of the first surgical robot 810 using a conversion matrix A₁₁ as illustrated in the following equation (9). Similarly, as illustrated in the following equation (10), the coordinate system (xᵥ, yᵥ, zᵥ) of the captured image of the observation device 800 can be converted into the coordinate system (xᵣ, yᵣ, zᵣ) of the second surgical robot 820 using a conversion matrix A₂₁. Since the positional relationship among the observation device 800, the first surgical robot 810, and the second surgical robot 820 is known, the conversion matrices A₁₁ and A₂₁ can be obtained.

[Equation 9]

(xᵣ₁,yᵣ₁,zᵣ₁,1)=(xᵥ,yᵥ,zᵥ,1)A₁₁ (9)

[Equation 10]

(xᵣ₂,yᵣ₂,zᵣ₂,1)=(xᵥ,yᵥ,zᵥ,1)A₂₁ (10)

On the other hand, since the configuration information of the first surgical robot 810 (the configuration information of each link and joint of the robot arm) and the configuration information of the surgical tool 811 attached to the distal end of the first surgical robot 810 are known, the positional relationship between the first surgical robot 810 and the surgical tool 811 is known. Similarly, the positional relationship between the second surgical robot 820 and the surgical tool 821 is known. Assuming that the coordinate system of the tip end of the surgical tool 811 is (xₑ₁, yₑ₁, zₑ₁) and the coordinate system of the tip end of the surgical tool 821 is (xₑ₂, yₑ₂, zₑ₂), as illustrated in the following equations (11) and (12), the coordinate system (xᵣ₁, yᵣ₁, zᵣ₁) of the first surgical robot 810 can be converted into the coordinate system (xₑ₁, yₑ₁, zₑ₁) of the tip end of the surgical tool 811 using a conversion matrix A₁₂, and the coordinate system (xᵣ₂, yᵣ₂, zᵣ₂) of the second surgical robot 820 can be converted into the coordinate system (xₑ₂, yₑ₂, zₑ₂) of the tip end of the surgical tool 811 using a conversion matrix A₂₂. The conversion matrix A₁₂ can be obtained on the basis of the configuration information of the first surgical robot 810 and the configuration information of the surgical tool 811, and the conversion matrix A₂₂ can also be obtained on the basis of the configuration information of the second surgical robot 820 and the configuration information of the surgical tool 821.

[Equation 11]

(xₑ₁,yₑ₁,zₑ₁,1)=(xᵣ₁,yᵣ₁,zᵣ₁,1)A₁₂ (11)

[Equation 12]

(xₑ₂,yₑ₂,zₑ₂,1)=(xᵣ₂,yᵣ₂,zᵣ₂,1)A₂₂ (12)

Therefore, the coordinate relationship between the captured image of the observation device 800 and the tip end of each of the surgical tools 811 and 812 is determined as illustrated in the following equations (13) and (14).

[Equation 13]

(xₑ₁,yₑ₁,zₑ₁,1)=(xᵥ,yᵥ,zᵥ,1)A₁₁A₁₂ (13)

[Equation 14]

(xₑ₂,yₑ₂,zₑ₂,1)=(xᵥ,yᵥ,zᵥ,1)A₂₁A₂₂ (14)

A case where the arrangement of the observation device 800, the first surgical robot 810, and the second surgical robot 820 as illustrated in FIG. 8 is applied to the surgical system 100 will be described. The captured image of the observation device 800 is displayed on the screen of the monitor display included in the presentation unit 113. The operator gives instruction on an operation amount of each of the surgical tools 811 and 812 on the captured image displayed on the monitor screen using the operation UI unit 112. The master-side control unit 111 transfers information on the amount of operation (xᵥ, yᵥ, zᵥ) expressed in the coordinate system of the captured image of the observation device 800 to the slave device 120 via the transmission path 130. Then, on the side of the slave device 120, in a case where the operation amount (xᵥ, yᵥ, zᵥ) is converted into the coordinate system (xₑ₁, yₑ₁, zₑ₁) of the tip end of the surgical tool 811 or the coordinate system (xₑ₁, yₑ₁, zₑ₁) of the tip end of the surgical tool 812 on the basis of the above equations (13) and (14), the operation amount is only required to be further converted into a command value of the first surgical robot 810 or the second surgical robot 820 (joint angle of each joint of the robot arm) for achieving the movement of the tip end of the surgical tool 811 or the surgical tool 812 corresponding to the operation amount of the operation UI unit 112 by inverse kinematics operation, to control the driving of the first surgical robot 810 or the second surgical robot 820.

As described above, also in the third example of the present disclosure, the operator views the captured image of the observation device 800 and performs the input to the operation UI unit 112, and the operation of the surgical tool 811 by the first surgical robot 810 and the operation of the surgical tool 821 by the second surgical robot 820 can be performed smoothly.

From the above equations (13) and (14), it can be seen that, according to the third example of the present disclosure, the conditional equation (4) described above in which the optimum hand-eye coordination is established is satisfied. Therefore, also in the third example of the present disclosure, even if the operator is not fully trained and proficient in hand-eye coordination, the operator can perform accurate surgery by making the observation device and the surgical system of the master-slave system cooperate with each other.

### F. EFFECTS

In this item F, effects brought about by applying the present disclosure to the surgical system 100 will be described.

According to the present disclosure, it is possible to provide a structure that highly accurately obtains a relative positional relationship between an observation device such as a microscope that observes an operative field and a surgical tool supported by a surgical robot. Therefore, it is possible to achieve precise manipulation, hand-eye coordination, and surgery support by cooperative operations of the observation device, the surgical robot, and further a plurality of surgical robots.

### INDUSTRIAL APPLICABILITY

The present disclosure is heretofore described in detail with reference to the specific embodiment. However, it is obvious that those skilled in the art can make modifications and substitutions of the embodiment without departing from the gist of the present disclosure.

In this description, the embodiment in which the surgical system according to the present disclosure is applied to ophthalmic surgery has been mainly described, but the gist of the present disclosure is not limited thereto. The present disclosure can be similarly applied to various types of surgical systems that support surgery using observation devices and surgical robots.

In short, the present disclosure is heretofore described in a form of an example and the content described in this specification should not be interpreted in a limited manner. In order to determine the gist of the present disclosure, the claims should be taken into consideration.

Note that the present disclosure can have the following configurations.
(1) A surgical system including:
an observation device that observes an operative field;
a surgical robot that supports a surgical tool;
a fixing portion that fixes a relationship of relative position and posture between the observation device and the surgical robot; and
a processing unit that performs processing for coordinate conversion between coordinates on a captured image of the observation device and coordinates of the surgical tool on the basis of the relationship of relative position and posture.

(2) The surgical system according to (1) described above, in which
the observation device includes a microscope and a pre-lens, and
the fixing portion that fixes a relationship of relative position and posture of the surgical robot with respect to the pre-lens.
(3) The surgical system according to (2) described above, in which
the processing unit converts coordinates on a captured image of the observation device into coordinates of the surgical tool using a synthesis matrix A₁A₂ obtained by synthesizing a first conversion matrix A₁ that converts coordinates on the captured image of the observation device into coordinates of the surgical robot and a second conversion matrix A₂ that converts coordinates of the surgical robot into coordinates of the surgical tool.
(4) The surgical system according to (1) described above, in which
the fixing portion fixes a relationship of relative position and posture of the surgical robot with respect to a retainer that has a marker and retains a state of the surgical site.
(5) The surgical system according to (4) described above, in which
the surgical site is an eyeball, and the retainer is an eyelid speculum.
(6) The surgical system according to (4) or (5) described above, in which
the processing unit converts the coordinates on the captured image of the observation device into the coordinates of the surgical tool using a synthesis matrix B₁B₂B₃ obtained by synthesizing a first conversion matrix B₁ that converts the coordinates on the captured image of the observation device into the coordinates of the retainer, a second conversion matrix B₂ that converts the coordinates of the retainer into the coordinates of the surgical robot, and a third conversion matrix B₃ that converts the coordinates of the surgical robot into the coordinates of the surgical tool.
(7) The surgical system according to (1) described above, including a first surgical robot and a second surgical robot that support surgical tools, respectively,
in which the observation device includes a microscope and a pre-lens, and
the fixing portion fixes a relationship of relative position and posture of each of the first surgical robot and the second surgical robot with respect to the pre-lens.
(8) The surgical system according to (7) described above, in which
the processing unit includes:
converting coordinates on a captured image of the observation device into coordinates of a surgical tool using a synthesis matrix A₁₁A₁₂ obtained by synthesizing a first conversion matrix A₁₁ that converts coordinates on the captured image of the observation device into coordinates of the first surgical robot and a second conversion matrix A₁₂ that converts coordinates of the surgical robot into coordinates of the surgical tool; and
converting coordinates on the captured image of the observation device into coordinates of the surgical tool using a synthesis matrix A₂₁A₂₂ obtained by synthesizing a first conversion matrix A₂₁ that converts coordinates on the captured image of the observation device into coordinates of the second surgical robot and a second conversion matrix A₂₂ that converts coordinates of the second surgical robot into coordinates of the surgical tool.
(9) The surgical system according to any one of (1) to (8) described above, in which
the processing unit converts an operation amount instructed on a captured image of the observation device into coordinates of the surgical tool.
(10) The surgical system according to (9) described above, further including
a control unit that controls driving of the surgical robot on the basis of the coordinates of the surgical tool converted by the processing unit.
(11) The surgical system according to any one of (1) to (10) described above, including
a master device that instructs an operation of the surgical robot, and a slave device that controls the operation of the surgical robot on the basis of the instruction from the master device.
(12) The surgical system according to (11) described above, in which
the master device inputs an instruction of an operation of the surgical tool by the operator on the captured image of the observation device,
the processing unit converts an operation amount instructed by the operator into coordinates of the surgical tool, and
the slave device performs an inverse kinematics operation on the coordinates of the surgical tool output from the processing unit to control an operation of the surgical robot.
(13) A surgical support method using a surgical system including an observation device that observes an operative field and a surgical robot that supports a surgical tool, a relationship of relative position and posture between the observation device and the surgical robot being fixed, the surgical support method including:
a processing step of performing processing for coordinate conversion between coordinates on a captured image of the observation device and coordinates of the surgical tool on the basis of the relationship of relative position and posture; and
a control step of controlling driving of the surgical robot on the basis of the coordinates of the surgical tool converted by the processing step.

### REFERENCE SIGNS LIST

100 Surgical system
110 Master device
111 Master-side control unit
112 Operation UI unit
113 Presentation unit
114 Master-side communication unit
120 Slave device
121 Slave-side control unit
122 Slave robot
123 Sensor unit
124 Slave-side communication unit
130 Transmission path
500 Observation device
501 Pre-lens
502 Fixing portion
510 Surgical robot
511 Surgical tool
600 Observation device
601 Pre-lens
602 Fixing portion
610 Surgical robot
611 Surgical tool
620 Eyelid speculum
621, 622, 623 Visual marker
800 Observation device
801 Pre-lens
802, 803 Fixing portion
810 First surgical robot
811 Surgical tool
820 First surgical robot
821 Surgical tool

1. A surgical system comprising:
an observation device that observes an operative field;
a surgical robot that supports a surgical tool;
a fixing portion that fixes a relationship of relative position and posture between the observation device and the surgical robot; and
a processing unit that performs processing for coordinate conversion between coordinates on a captured image of the observation device and coordinates of the surgical tool on a basis of the relationship of relative position and posture.

2. The surgical system according to claim 1,
wherein the observation device includes a microscope and a pre-lens, and the fixing portion that fixes a relationship of relative position and posture of the surgical robot with respect to the pre-lens.

3. The surgical system according to claim 2,
wherein the processing unit converts coordinates on a captured image of the observation device into coordinates of the surgical tool using a synthesis matrix A₁A₂ obtained by synthesizing a first conversion matrix A₁ that converts coordinates on the captured image of the observation device into coordinates of the surgical robot and a second conversion matrix A₂ that converts coordinates of the surgical robot into coordinates of the surgical tool.

4. The surgical system according to claim 1,
wherein the fixing portion fixes a relationship of relative position and posture of the surgical robot with respect to a retainer that has a marker and retains a state of the surgical site.

5. The surgical system according to claim 4,
wherein the surgical site is an eyeball, and the retainer is an eyelid speculum.

6. The surgical system according to claim 4,
wherein the processing unit converts the coordinates on the captured image of the observation device into the coordinates of the surgical tool using a synthesis matrix B₁B₂B₃ obtained by synthesizing a first conversion matrix B₁ that converts the coordinates on the captured image of the observation device into the coordinates of the retainer, a second conversion matrix B₂ that converts the coordinates of the retainer into the coordinates of the surgical robot, and a third conversion matrix B₃ that converts the coordinates of the surgical robot into the coordinates of the surgical tool.

7. The surgical system according to claim 1, comprising a first surgical robot and a second surgical robot that support surgical tools, respectively,
wherein the observation device includes a microscope and a pre-lens, and
the fixing portion fixes a relationship of relative position and posture of each of the first surgical robot and the second surgical robot with respect to the pre-lens.

8. The surgical system according to claim 7,
wherein the processing unit includes:
converting coordinates on a captured image of the observation device into coordinates of a surgical tool using a synthesis matrix A₁₁A₁₂ obtained by synthesizing a first conversion matrix A₁₁ that converts coordinates on the captured image of the observation device into coordinates of the first surgical robot and a second conversion matrix A₁₂ that converts coordinates of the surgical robot into coordinates of the surgical tool; and
converting coordinates on the captured image of the observation device into coordinates of the surgical tool using a synthesis matrix A₂₁A₂₂ obtained by synthesizing a first conversion matrix A₂₁ that converts coordinates on the captured image of the observation device into coordinates of the second surgical robot and a second conversion matrix A₂₂ that converts coordinates of the second surgical robot into coordinates of the surgical tool.

9. The surgical system according to claim 1,
wherein the processing unit converts an operation amount instructed on a captured image of the observation device into coordinates of the surgical tool.

10. The surgical system according to claim 9,
further comprising a control unit that controls driving of the surgical robot on a basis of the coordinates of the surgical tool converted by the processing unit.

11. The surgical system according to claim 1, comprising
a master device that instructs an operation of the surgical robot, and a slave device that controls the operation of the surgical robot on a basis of the instruction from the master device.

12. The surgical system according to claim 11,
wherein the master device inputs an instruction of an operation of the surgical tool by the operator on the captured image of the observation device,
the processing unit converts an operation amount instructed by the operator into coordinates of the surgical tool, and
the slave device performs an inverse kinematics operation on the coordinates of the surgical tool output from the processing unit to control an operation of the surgical robot.

13. A surgical support method using a surgical system including an observation device that observes an operative field and a surgical robot that supports a surgical tool, a relationship of relative position and posture between the observation device and the surgical robot being fixed, the surgical support method comprising:
a processing step of performing processing for coordinate conversion between coordinates on a captured image of the observation device and coordinates of the surgical tool on a basis of the relationship of relative position and posture; and
a control step of controlling driving of the surgical robot on a basis of the coordinates of the surgical tool converted by the processing step.
